(19) United States
(12) Patent Application Publication
(10) Pub. No.: US 2014/0187996 A1
(43) Pub. Date: Jul. 3, 2014

(54) Electrodermal Measurement System And Method

(71) Applicants: Evian Gordon, Vaucluse (AU); Peter Maul, Figtree (AU)

(72) Inventors: Evian Gordon, Vaucluse (AU); Peter Maul, Figtree (AU)

(21) Appl. No.: 14/148,291

(22) Filed: Jan. 6, 2014

Related U.S. Application Data
(63) Continuation of application No. PCT/AU2012/000811, filed on Jul. 6, 2012.

(30) Foreign Application Priority Data
Jul. 6, 2011 (AU) 2011902693

(51) Int. Cl. A61B 5/053

(52) U.S. Cl. CPC A61B 5/053
USPC 600/547

(57) ABSTRACT

A method of determining the conductance of the skin of a human or animal, the method including the step of: (a) eliminating any equivalent parasitic voltages in the skin by taking multiple different measurements of the conductance utilizing different probe voltage levels, (b) calculating the conductance from the sampled results of the multiple different measurements.

### FIELD OF THE INVENTION

The present invention relates to the measurement of electrodermal activity of the skin. In particular, the present invention discloses a system and method for highly accurate electrodermal activity measurement.

### BACKGROUND OF THE INVENTION

Any discussion of the background art throughout the specification should in no way be considered as an admission that such art is widely known or forms part of common general knowledge in the field.

Electrodermal activity of the skin is normally measured by taking a resistance measurement of the skin. Accurate measurement of actual fluctuations in skin resistance is plagued by many problems as a parasitic voltage can be present as a result of the contact of a metal sensing probe on the skin.

The parasitic voltage can have a fluctuating magnitude which is difficult to determine from time to time and is therefore difficult to compensate for. There remains a general need for the accurate measurement of actual electrodermal activity.

### SUMMARY OF THE INVENTION

It is an object of the present invention to provide a useful alternative system and method for the accurate measurement of electrodermal activity.

In accordance with a first aspect of the present invention, there is provided a method of determining the conductance of the skin of a human or animal, the method including the steps of: (a) eliminating any equivalent parasitic voltages in the skin by taking multiple different measurements of the conductance utilizing different probe voltage levels, and (b) calculating the conductance from the sampled results of the multiple different measurements.

In accordance with a further aspect of the present invention, there is provided a method of measuring conductance of a human or animal body, the method including the steps of: (a) interconnecting a first circuit to a grounded body, the first circuit including a first known resistance R1 connected in series with the human body; (b) applying a first voltage E1 to the first circuit; (c) measuring a voltage drop V1 across the first known resistance; (d) applying a second voltage E2 to the first circuit; (e) measuring a voltage drop V2 across the first known resistance; and (f) calculating a conductance S substantially in accordance with the equation:

$$S = \frac{( V 2 - V 1 )}{R 1 ( E 2 - E 1 ) - ( V 2 - V 1 )}$$

In some embodiments, the steps (a) to (f) are preferably repeated in an iterative manner with the conductance tracked over time. The repetition rate can be greater than 100 Hertz. In some embodiments, the resistance R1 can be substantially 100K Ohms.

In accordance with a further aspect of the present invention, there is provided an apparatus for measuring the conductance of a human or animal body, the apparatus including: a first circuit including a first known resistance R1 connected in series with the human body; a first voltage switchable source E1 interconnected to the first circuit; a second voltage switchable source E2 connected to the first circuit; a voltage detector measuring the voltage across resistance R1; a controller interconnected to the voltage detector and the first and second switchable source, the controller switching a first source voltage E1 into the first circuit and the voltage detector measuring the voltage V1 across R1, the controller then switching a second source voltage E2 and measuring a second voltage V2 across the resistor R1, the controller than calculating a conductance S substantially in accordance with the equation:

$$S = \frac{( V 2 - V 1 )}{R 1 ( E 2 - E 1 ) - ( V 2 - V 1 )}$$

### BRIEF DESCRIPTION OF THE DRAWINGS

Preferred embodiments of the invention will now be described, by way of example only, with reference to the accompanying drawings in which:

FIG. 1 illustrates schematically a first switching circuit for measuring skin conductance; and

FIG. 2 illustrates schematically the extension of the arrangement of FIG. 1 to an iterative switching circuit.

### DETAILED DESCRIPTION OF THE INVENTION

In the preferred embodiments there is provided a means for eliminating the influence of parasitic voltage V in skin conductance measurements.

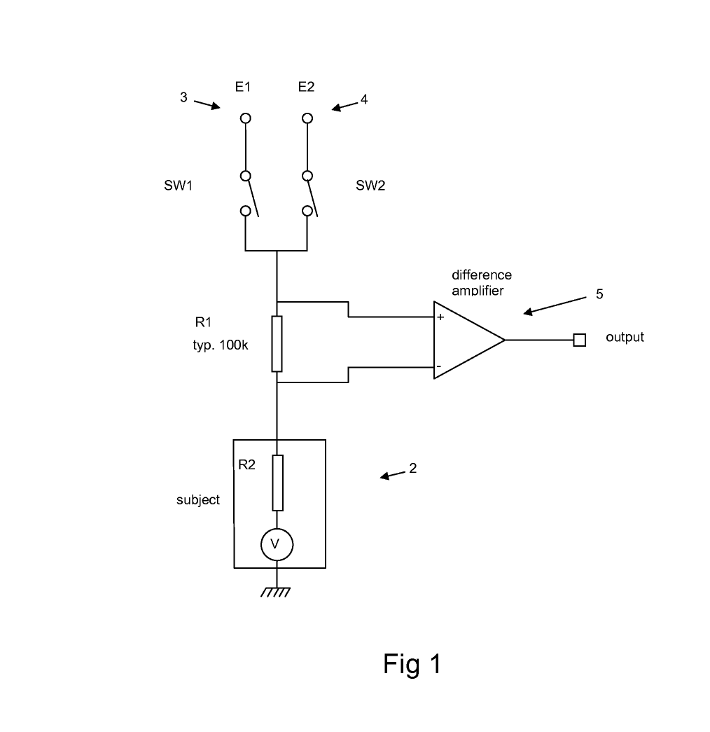

Turning initially to FIG. 1 there is illustrated schematically an initial circuit diagram instructive of the teachings of the preferred embodiment.

As will be evident to the skilled artesian, the grounded human body can be represented 2 by an equivalent circuit including resistance R2 and parasitic voltage v. The preferred embodiment proceeds by applying two voltages E1 and E2 in succession via switches SW1 and SW2 and measures a voltage across a defined resistance R1. The voltage across a defined resistance R1, which is typically a 100K Ohm resistance, is forwarded to a difference amplifier 5 and the resulting output measured.

By applying the two voltages alternately and measuring the two respective currents, v can be eliminated. The proposed method has multiple stages, rather like an internal combustion engine. It alternately switches different resistive circuits and measures the corresponding voltage drop across the resistor R1. The steps can proceed as follows:

1. Switch off E2, Switch on E1
2. Measure output voltage and assign to V1
3. Switch off E1, Switch on E2
4. Measure output voltage and assign to V2
5. Apply formula below to calculate conductance S and repeat

The resulting conductance S can be determined by the following formula:

$$S = \frac{( V 2 - V 1 )}{R 1 ( E 2 - E 1 ) - ( V 2 - V 1 )}$$

The unwanted voltage v is cancelled and does not form part of the equation. S, the conductance, can be measured and scaled to represent a value in microSiemens. A typical industry scale is 1 millivolt per 10 microSiemens.

In one example arrangement, E1 can be 5 volts and E2 can be 12 volts. Other voltage ranges can be utilized. However, for numerical stability, the two voltages should be dissimilar.

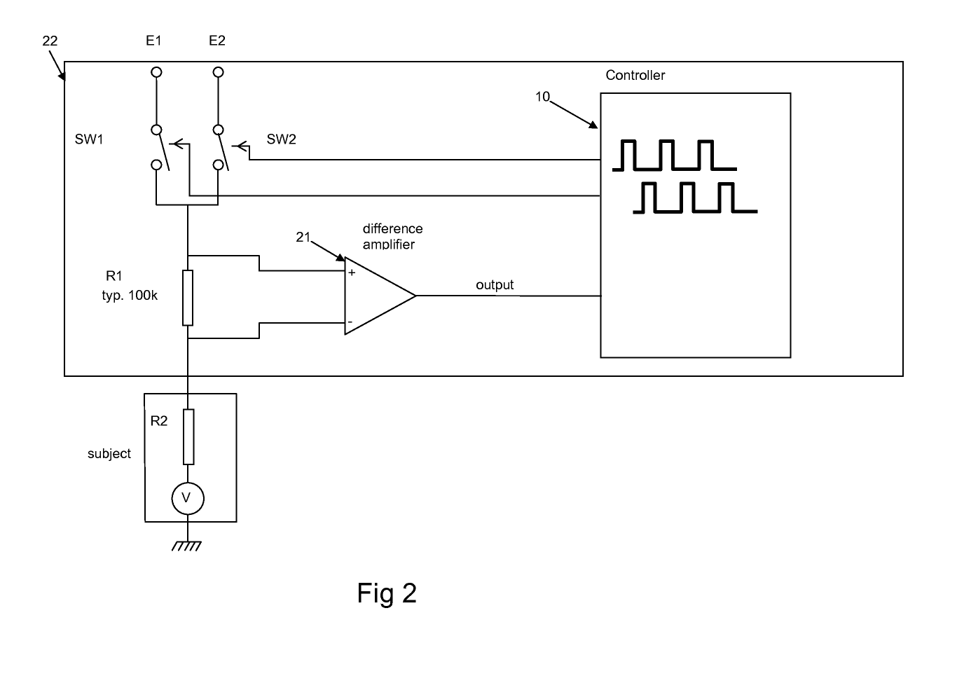

The arrangement of FIG. 1 can be extended in an automated manner as illustrated schematically in FIG. 2. In this case the switches SW1 and SW2 are electronic switches that switch the two excitation voltages E1 and E2 onto the subject. The switches act under the control of a microcontroller 10. They are switched one after another in a clocked manner and the amplifier 21 outputs the voltage across the resistor R1 in a clocked manner. The output is fed back into the microcontroller 10 for sampling. The switching frequency is not critical, but should be low enough to not introduce noise and high enough to not produce cogging. It has been found that a switching frequency of a few kilohertz works well.

The arrangement of FIG. 2 has been found to be useful for measuring the surprise response in people. Ideally, the arrangement can be constructed as a portable device 22 and the readings can be automatically taken and stored whilst the person is not stationary. The readings being later downloaded for analysis.

Interpretation

The following description and figures make use of reference numerals to assist the addressee understand the structure and function of the embodiments. Like reference numerals are used in different embodiments to designate features having the same or similar function and/or structure.

The drawings need to be viewed as a whole and together with the associated text in this specification. In particular, some of the drawings selectively omit including all features in all instances to provide greater clarity about the specific features being described. While this is done to assist the reader, it should not be taken that those features are not disclosed or are not required for the operation of the relevant embodiment.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of exemplary embodiments of the invention, various features of the invention are sometimes grouped together in a single embodiment, Fig., or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

What is claimed is:

1. A method of determining the conductance of the skin of a human or animal, the method including the step of:
(a) eliminating any equivalent parasitic voltages in the skin by taking multiple different measurements of the conductance utilizing different probe voltage levels,
(b) calculating the conductance from the sampled results of the multiple different measurements.

2. The method as claimed in claim 1 wherein the number of different probe voltage levels is two.

3. A method of measuring conductance of a human or animal body, the method including the steps of:
(a) interconnecting a first circuit to a grounded body, the first circuit including a first known resistance R1 connected in series with the human body;
(b) applying a first voltage E1 to the first circuit;
(c) measuring a voltage drop V1 across the first known resistance;
(d) applying a second voltage E2 to the first circuit;
(e) measuring a voltage drop V2 across the first known resistance; and
(f) calculating a conductance S substantially in accordance with the equation:
$$S = \frac{( V 2 - V 1 )}{R 1 ( E 2 - E 1 ) - ( V 2 - V 1 )} .$$

4. The method as claimed in claim 3 wherein the steps (a) to (f) are repeated in an iterative manner with the conductance tracked over time.

5. The method as claimed in claim 4 wherein the repetition rate is greater than 100 Hertz.

6. The method as claimed in claim 3 wherein resistance R1 is substantially 100K Ohms.

7. An apparatus for measuring the conductance of a human or animal body, the apparatus including:
a first circuit including a first known resistance R1 connected in series with the human body;
a first voltage switchable source E1 interconnected to the first circuit;
a second voltage switchable source E2 connected to the first circuit;
a voltage detector measuring the voltage across resistance R1;
a controller interconnected to said voltage detector and said first and second switchable source, the controller switching a first source voltage E1 into the first circuit and the voltage detector measuring the voltage V1 across R1, the controller then switching a second source voltage E2 and measuring a second voltage V2 across the resistor R1, the controller than calculating a conductance S substantially in accordance with the equation:
$$S = \frac{( V 2 - V 1 )}{R 1 ( E 2 - E 1 ) - ( V 2 - V 1 )} .$$

8. The apparatus as claimed in claim 7 wherein said controller interconnects source voltages E1, E2 and calculates the corresponding conductance S in an iterative manner.
